(19) United States
(12) Patent Application Publication
(10) Pub. No.: US 2016/0361291 A1
(43) Pub. Date: Dec. 15, 2016

(54) METHODS FOR INCREASING SKELETAL MUSCLE PROTEIN SYNTHESIS USING GREEN TEA EXTRACT

(71) Applicant: ABBOTT LABORATORIES, Abbott Park, IL (US)

(72) Inventors: BENJAMIN MEADOR, Monticello, IL (US); SUZETTE PEREIRA, Westerville, OH (US); NEILE EDENS, Columbus, OH (US); MICHAEL TISDALE, Birmingham (GB)

(73) Assignee: ABBOTT LABORATORIES, Abbott Park, IL (US)

(21) Appl. No.: 15/105,165

(22) PCT Filed: Dec. 8, 2014
(86) PCT No.: PCT/US2014/069126

§ 371 Date: Jun. 16, 2016

Related U.S. Application Data
(60) Provisional application No. 61/917,763, filed on Dec. 18, 2013.

(51) Int. Cl. A61K 31/353; A61K 9/00; A23L 33/115; A23L 33/105; A23L 33/17; A23L 33/125; A61K 36/82; A23L 33/00

(52) U.S. Cl. CPC A61K 31/353; A61K 36/82; A61K 9/0053; A23L 33/30; A23L 33/105; A23L 33/17; A23L 33/125; A23L 33/115; A23V 2002/00

(57) ABSTRACT

Methods of increasing skeletal muscle protein synthesis in a subject are provided. Methods of increasing or maintaining mammalian target of rapamycin (mTOR) activation are also provided. Such methods include a step of administering at least one serving per day of a composition including 20 to 2,000 mg of a green tea extract per serving to the subject.

### CROSS REFERENCE TO RELATED APPLICATIONS

This application claims priority to and any benefit of U.S. Provisional Application No. 61/917,763, filed Dec. 18, 2013, the entire contents of which are incorporated by reference in its entirety.

### FIELD OF THE DISCLOSURE

The present disclosure relates to methods of increasing skeletal muscle protein synthesis in a subject. Particularly, the present disclosure relates to the use of compositions comprising green tea extract to increase skeletal muscle protein synthesis in such subject.

### BACKGROUND

Skeletal muscle loss in a subject due to age-, inactivity-, or disease-related disorders may have a negative impact on the overall health and well-being of the subject. For example, skeletal muscle loss may lead to loss of muscle function, weakness, frailty, further muscle loss, susceptibility to injury, a decrease in the ability or desire to exercise, and so forth in the subject. Typically, skeletal muscle loss results from an imbalance in the rates of the skeletal muscle protein synthesis and degradation. For example, muscle wasting due to sarcopenia, muscle disuse, or immobilization is a result of a drastic reduction of protein synthesis in skeletal muscles. Such a drastic reduction in protein synthesis disrupts the normal equilibrium between protein synthesis and protein degradation required for maintaining muscle mass and function.

From a nutritional perspective, subjects can endeavor to mitigate skeletal muscle loss, or for healthy subjects increase skeletal muscle mass, by consuming nutritional products containing a balance of protein, carbohydrates, vitamins, minerals, and other ingredients. However, the number of nutritional ingredients known to be capable of impacting skeletal muscle protein balance, particularly for increasing skeletal muscle protein synthesis, is limited. Ingredients such as milk proteins, branched chain amino acids (BCAAs), and essential amino acids may be effective, but may not be practical because they have to be used in high doses to show efficacy. For example, the required amounts of such proteins and amino acids limit their application in certain types of nutritional products (e.g., clear nutritional beverages) due to product formulation issues such as cloudiness, sedimentation, etc. In addition, free BCAAs have organoleptic issues making them infeasible, or at the least, undesirable for a nutritional product.

### SUMMARY

Disclosed herein are methods of increasing skeletal muscle protein synthesis in a subject. The muscle protein synthesis is increased by administering green tea extract to the subject.

In accordance with certain embodiments, methods of increasing skeletal muscle protein synthesis in a subject are disclosed. The methods include administering at least one serving per day of a composition including 20 milligrams (mg) to 2,000 mg of a green tea extract per serving to the subject. The administration of the green tea extract to the subject is effective to increase skeletal muscle protein synthesis in the subject.

Certain embodiments also include a composition for use in increasing skeletal muscle protein synthesis in a subject wherein the composition comprises 20 mg to 2,000 mg of a green tea extract per serving and wherein the dosage is at least one serving per day.

Certain embodiments are also directed to use of a composition in the manufacture of a medicament for use in increasing skeletal protein synthesis in a subject, the composition comprising 20 mg to 2,000 mg of a green tea extract, wherein the dosage is at least one serving a day.

In addition, according to certain embodiments, methods of increasing or maintaining mammalian target of rapamycin (mTOR) activation in a subject in need thereof are disclosed. The methods include administering at least one serving per day of a composition including 20 mg to 2,000 mg of a green tea extract per serving to the subject. The administration of the green tea extract to the subject is effective to increase skeletal muscle protein synthesis in the subject.

Certain embodiments also include a composition for use in increasing or maintaining mTOR activation in a subject in need thereof wherein the composition comprises 20 mg to 2,000 mg of a green tea extract per serving and wherein the dosage is at least one serving per day.

Certain embodiments are also directed to use of a composition in the manufacture of a medicament for use in increasing or maintaining mTOR activation in a subject in need thereof wherein the composition comprises 20 mg to 2,000 mg of a green tea extract per serving and wherein the dosage is at least one serving per day.

### BRIEF DESCRIPTION OF THE DRAWING

FIG. 1 shows the effect of epigallocatechin gallate (EGCg), in the form of green tea extract, on total protein synthesis in C₂C₁₂ myotubes in the presence of tumor necrosis factor alpha (TNF-α).

FIG. 2 shows the effect of EGCg, in the form of green tea extract, on mTOR activation of Sprague Dawley rats at the conclusion of an eight week study in which the rats were fed a control diet and a diet containing the EGCg.

FIG. 3 shows the effect of EGCg, in the form of green tea extract, on gastrocnemius muscle mass of Sprague Dawley rats at the conclusion of an eight week study in which the rats were fed a control diet and a diet containing the EGCg.

FIG. 4 shows the effect of EGCg, in the form of green tea extract, on muscle fiber cross sectional area of Sprague Dawley rats at the conclusion of an eight week study in which the rats were fed a control diet and a diet containing the EGCg.

FIG. 5 shows the effect of EGCg, in the form of green tea extract, on 19S regulatory particle ubiquitin proteasome subunits of Sprague Dawley rats at the conclusion of an eight week study in which the rats were fed a control diet and a diet containing the EGCg.

FIG. 6 shows the effect of EGCg, in the form of green tea extract, on 20S core particle ubiquitin proteasome subunits of Sprague Dawley rats at the conclusion of an eight week study in which the rats were fed a control diet and a diet containing the EGCg.

FIG. 7 shows the effect of EGCg, in the form of green tea extract, on muscle ring finger protein 1 (MuRF1) of Sprague Dawley rats at the conclusion of an eight week study in which the rats were fed a control diet and a diet containing the EGCg.

FIG. 8 shows the effect of EGCg, in the form of green tea extract, on muscle atrophy f-box (MAFbx) of Sprague Dawley rats at the conclusion of an eight week study in which the rats were fed a control diet and a diet containing the EGCg.

### DETAILED DESCRIPTION

The term “administering,” unless otherwise indicated herein, should be understood to include providing the nutritional product to a subject, the act of consuming the nutritional product, and combinations thereof.

The term “nutritional composition” as used herein, unless otherwise specified, refers to nutritional products in various forms including, but not limited to, liquids, solids, powders, semi-solids, semi-liquids, nutritional supplements, and any other nutritional food product known in the art. A nutritional composition in powder form may be reconstituted upon addition of water or another liquid to form a liquid nutritional composition prior to administration to (e.g., providing to or consumption by) a subject. As discussed below, in certain embodiments disclosed herein, the nutritional compositions comprise at least one of a source of protein, a source of carbohydrate, and a source of fat. The nutritional compositions disclosed herein are generally suitable for oral consumption by a human.

The term “liquid nutritional composition” as used herein, unless otherwise specified, refers to nutritional compositions in ready-to-drink liquid form, e.g., liquid nutritional products; concentrated liquid form; and nutritional liquids made by reconstituting nutritional powders as described herein prior to use. The liquid nutritional composition may also be formulated as a suspension, an emulsion, a solution, and the like.

The term “nutritional powder” or “reconstitutable powder” as used herein, unless otherwise specified, refers to nutritional compositions in a solid flowable or scoopable form that can be reconstituted with water or another liquid prior to consumption and includes spray-dried powders, dry-mixed, or dry-blended powders, and the like.

The term “nutritional semi-solid” as used herein, unless otherwise specified, refers to nutritional products that are intermediate in properties, such as rigidity, between solids and liquids. Some semi-solid examples include puddings, yogurts, gels, gelatins, doughs, and the like.

The term “nutritional semi-liquid” as used herein, unless otherwise specified, refers to nutritional compositions that are intermediate in properties, such as flow properties, between liquids and solids. Some semi-liquid examples include thick shakes, liquid yogurts, liquid gels, and the like.

The term “serving,” unless otherwise indicated herein, generally refers to an amount of nutritional composition that is intended for consumption or otherwise consumed in one sitting, which may last up to one or up to two hours.

The term “subject,” unless otherwise indicated herein, refers to a mammal, including but not limited to, a human, a domesticated farm animal (e.g., cow, horse, pig), or a pet (e.g., dog, cat). In certain embodiments disclosed herein, the subject is a human.

Disclosed herein are methods of increasing skeletal muscle protein synthesis in a subject. The muscle protein synthesis is increased by administering green tea extract to the subject. In particular, the methods comprise administering at least one serving per day of a composition including 20 mg to 2,000 mg of a green tea extract per serving to the subject. The administration of the compositions containing green tea extract is effective to increase skeletal muscle protein synthesis in the subject. In other words, the compositions disclosed herein are for use in increasing skeletal muscle protein synthesis in a subject, by administering at least one serving per day of a composition including 20 mg to 2,000 mg of a green tea extract per serving to the subject.

In accordance with certain embodiments of the methods disclosed herein, the subject is susceptible to skeletal muscle loss. Subjects susceptible to skeletal muscle loss include those who experience skeletal muscle loss, those who are at risk of skeletal muscle loss, and combinations of both those who experience and are at risk of skeletal muscle loss. Such skeletal muscle loss may be due to age, malnourishment, disease, injury, infection, hospitalization, lack of appetite, lack of mobility, medication, and combinations thereof.

In accordance with certain embodiments disclosed herein, the skeletal muscle loss can be characterized as age-related skeletal muscle loss, such as the skeletal muscle loss due to at least one of sarcopenia, hospitalization, surgery, post-hospitalization rehabilitation, appetite loss, inflammation, dysphagia, cognitive impairment, impaired nutrient absorption, taste aversion, frailty syndrome, and combinations thereof. In accordance with the preceding and other embodiments, the skeletal muscle loss can be characterized as disease-related skeletal muscle loss, such as skeletal muscle loss due to at least one of cancer cachexia, chronic obstructive pulmonary disease (COPD), end stage renal disease (ESRD), congestive heart failure (CHF), acquired immunodeficiency syndrome (AIDS), chemotherapy, medications, acute illness, and combinations thereof. In accordance with the preceding and yet other embodiments, the skeletal muscle loss can be characterized as atrophy-related skeletal muscle loss, such as skeletal muscle loss due to at least one of lack of mobility, lack of use of extremities, disability, muscular dystrophy, joint disease, and combinations thereof. One skilled in the art would understand that the different forms of skeletal muscle loss disclosed herein, e.g., age-related, disease-related, atrophy-related, may not be mutually exclusive and may overlap in scope.

As mentioned above, skeletal muscle loss results from an imbalance in the respective rates of the skeletal muscle protein synthesis and skeletal muscle protein degradation. Protein synthesis within the muscle is critical for normal function, contributing to the replacement of damaged proteins, accrual of cytoskeletal proteins (hypertrophy), and the production of intra- and extra-cellular signaling compounds. In accordance with or in addition to the preceding embodiments, the skeletal muscle loss is caused by a condition or agent that inhibits skeletal muscle protein synthesis. Accordingly, in certain embodiments of the methods disclosed herein, at least a portion of the at least one condition or agent that inhibits muscle protein synthesis may result from age, malnourishment, disease, injury, infection, hospitalization, lack of appetite, lack of mobility, and any of the skeletal muscle loss characterized above as age-related, disease-related, or atrophy-related skeletal muscle loss, the like, and combinations thereof. One skilled in the art would understand that the conditions or agents disclosed herein may not be mutually exclusive and may overlap in scope with other causes of skeletal muscle loss disclosed herein. Examples of such conditions or agents include, but are not limited to, conditions such as starvation, stress, decrease in sex hormones, metabolic syndrome, insulin resistance, inflammation, renal dysfunction, and the like; inflammatory agents such as interleukin 6 (IL-6), tumor necrosis factor alpha (TNF-α), and the like; agents including bacteria-based toxins such as lipopolysaccharides; other types of agents such as angiotensin, proteolysis inducing factors (PIF), myostatin, glucocorticoids, and the like; and combinations thereof.

In accordance with certain of the preceding embodiments of the methods disclosed herein, by administering the composition, the skeletal muscle protein synthesis in the subject increases under stress caused by at least one condition or agent that inhibits skeletal muscle protein synthesis.

In accordance with certain other embodiments of the methods disclosed herein, the subject is not susceptible to or experiencing skeletal muscle loss. In other words, at least with respect to skeletal muscle loss, the subject is healthy (e.g., the subject has or maintains a healthy balance in the rates of the skeletal muscle protein synthesis and degradation). In such embodiments, the methods disclosed herein are beneficial to maintaining or gaining skeletal muscle mass (lean body mass) in the subject. Furthermore, the methods disclosed herein can be used alone, or in combination with balanced nutrition and exercise to maintain or gain skeletal muscle mass.

As mentioned above, the composition administered to the subject includes 20 mg to 2,000 mg of a green tea extract per serving. In accordance with this and other embodiments, the composition includes 40 mg to 2,000 mg of green tea extract per serving, including from 100 mg to 2,000 mg, from 250 mg to 2,000 mg, from 500 mg to 2,000 mg, from 20 mg to 1,500 mg, from 40 mg to 1,500 mg, from 100 mg to 1,500 mg, and from 250 mg to 1,500 mg of green tea extract per serving.

The green tea extracts disclosed herein are derived from green tea leaves, in which the polyphenol epigallocatechin gallate (“EGCg”) alone, or in combination with other polyphenol compounds, are isolated from green tea as an extract. Examples of such suitable green tea extracts are in the form of a liquid, a solid (e.g., a powder), and mixtures thereof. In some embodiments, the green tea extract may be decaffeinated, or in other words, substantially free of caffeine. As used herein, “decaffeinated” or “substantially free of caffeine” refers to green tea extract that contains less than 1% by weight solids of caffeine based on the total weight of the extract, including less than 0.5%, less than 0.1%, and less than 0.01% by weight solids of caffeine based on the total weight of the extract. The green tea extract disclosed herein may also contain no caffeine, i.e., zero caffeine.

In addition to containing EGCg, suitable green tea extracts used with the nutritional compositions disclosed herein may contain other polyphenols including but not limited to, other catechins such as catechin (i.e., (+)-catechin, also known as “C”), epicatechin (“EC”), gallocatechin (“GC”), epigallocatechin (“EGC”), and epicatechin gallate (“ECg”); flavones such as apigenin, isoviloxin, sapotarin, and vicenin-2; flavonols such as kaempherol, quercetin, and myricetin; condensed flavanoids, and tannin glycosides. Accordingly, in certain embodiments, in addition to the EGCg, the green tea extracts disclosed herein include at least one of C, EC, GC, EGC, ECg, and combinations thereof.

In accordance with certain embodiments disclosed herein, the green tea extract contains at least 30% by weight solids of EGCg, including at least 45%, at least 50%, at least 70%, at least 80%, at least 90%, and at least 100% by weight solids of EGCg. In accordance with certain of the preceding embodiments, the green tea extract contains 30% to 100% by weight solids of EGCg, including from 45% to 100%, 50% to 100%, 70% to 100%, 80% to 100%, and 90% to 100% by weight solids of EGCg.

In accordance with certain embodiments of the methods disclosed herein, the administration of the composition containing green tea extract, particularly EGCg, increases or maintains mTOR activation in the subject. In accordance with such embodiments, the methods comprise administering at least one serving per day of a composition including 20 mg to 2,000 mg of a green tea extract per serving to the subject. The administration of the composition is effective to increase or maintain mTOR activation in the subject, thereby increasing skeletal muscle protein synthesis. In other words, the compositions containing green tea extract improves or preserves mTOR activation in the subject, thereby increasing muscle protein synthesis. mTOR is a protein kinase that regulates protein synthesis and cell growth in skeletal muscle and other tissue types. As shown in Example 2, gastrocnemius muscle wet weight and muscle fiber cross sectional area increased in rats experiencing sarcopenia that were fed green tea extract (including EGCg). These increases are all associated with an increased mTOR activation. Conversely, in this Example, the muscle ring-finger protein 1 (MuRF1) and muscle atrophy f-box (MAFbx), which are markers of skeletal muscle atrophy, decreased in the rats fed the green tea extract (including EGCg). In accordance with certain embodiments of the methods disclosed herein, the subject is in need of increasing or maintaining mTOR activation. Thus, in accordance with the preceding and other embodiments, methods for increasing or maintaining mTOR activation in a subject in need thereof are provided herein. Such methods include administering at least one serving per day of the compositions including 20 mg to 2,000 mg of a green tea extract per serving to the subject.

Alternatively or in addition, in accordance with certain embodiments disclosed herein, the green tea extract may contain at least 3% to 20% by weight solids of EC, including from 4% to 15%, and from 5% to 10% by weight solids of EC. In accordance with certain embodiments, the EC content of the compositions disclosed herein may be fortified or supplemented by including sources of EC other than green tea extract, such as cocoa.

The green tea extract is formulated into a suitable composition and then, in accordance with the methods disclosed herein, administered to a subject in a form adapted to the chosen route of administration. In accordance with certain embodiments, the compositions disclosed herein include, but are not limited to, those suitable for oral administration. Oral administration, as defined herein, includes any form of administration in which the composition including the green tea extract passes through the esophagus of the subject. For example, oral administration typically refers to oral consumption, but may also include administration through nasogastric intubation, in which a tube is run from the nose to the stomach of the subject to administer the composition. Oral administration is a form of enteral administration (i.e., administration through the digestive track). Other forms of enteral administration suitable for use with the methods disclosed herein include administration through a gastric or jejunal tube. In accordance with the embodiments described herein, suitable forms of the composition for enteral administration to the subject include caplets, tablets, pills, capsules, chewable tablets, quick dissolve tablets, effervescent tablets, solutions, suspensions, emulsions, multi-layer tablets, bi-layer tablets, soft gelatin capsules, hard gelatin capsules, lozenges, chewable lozenges, beads, granules, particles, microparticles, dispersible granules, sachets, and combinations thereof.

In certain embodiments, the compositions may be formulated consisting of or consisting essentially of green tea extract. In other embodiments, the compositions containing the green tea extract are formulated as a nutritional composition. Such nutritional compositions disclosed herein are useful to provide supplemental, primary, or sole sources of nutrition, including providing the subjects one or more benefits as described herein. In certain embodiments, the nutritional composition provides up to 500 kilocalories (kcal) of energy per serving or dose, including from 20 kcal to 500 kcal, from 75 kcal to 500 kcal, from 150 kcal to 500 kcal, from 250 kcal to 500 kcal, from 300 kcal to 500 kcal, or from 400 kcal to 500 kcal per serving.

In addition to the green tea extract, the nutritional compositions comprise at least one of a source of protein, a source of carbohydrate, a source of fat, and combinations thereof. In accordance with certain methods of the embodiments disclosed herein, the nutritional compositions are provided as needed to supply the desired level of green tea extract, including providing at least one serving per day to achieve the desired effect. The foregoing should be understood to include, but not be limited to, one serving per day, two servings per day, three servings per day, four servings per day, etc. Typically the compositions disclosed herein are administered in at least one serving per day or at least two servings per day. In other embodiments, the compositions disclosed herein are administered continuously or intermittently up to 24 hr/day by infusion through a nasogastric, gastric, or jejeunal feeding tube.

The compositions including the green tea extract disclosed herein can also be referred to herein as medicaments. For example, in accordance with the embodiments disclosed herein, green tea extract can be used for the preparation or manufacture of a medicament for treating a subject susceptible to unintentional weight loss by maintaining or increasing bodyweight.

In certain embodiments, the compositions disclosed herein are administered to the subject for at least 3 days. In accordance with the preceding and other embodiments, the compositions disclosed herein can be administered to a subject for at least 1 week, for at least 10 days, for at least 2 weeks, for at least 1 month, for at least 6 months, for at least 1 year, or for more than one year. Within the context of administering a serving to a subject, the number of days is intended to reflect the days in which a subject has been instructed to be administered the composition, and in which the composition is actually administered for at least 65%, including at least 90%, of the instructed days during the period of administration. In accordance with certain embodiments, when the subject is susceptible to, or in some manner is enduring, acute skeletal muscle loss, e.g., loss that is the result of, related to, or, alternatively, is subsequent to, hospitalization or intensive care (ICU), the compositions disclosed herein are administered for at least 3 days, including from 3 to 10 days. In other embodiments, when the subject is susceptible to, or in some manner is enduring, chronic skeletal muscle loss, the compositions disclosed herein are administered for greater than 10 days, including from greater than 10 days up to 14 days, from greater than 10 days up to 21 days, from greater than 10 days up to 1 month, from greater than 10 days up to 6 months, and from greater than 10 days up to 1 year, or for greater than 10 days to longer than 1 year.

In accordance with certain embodiments, the compositions disclosed herein containing the green tea extract are nutritional compositions. The nutritional compositions are formulated as, and intended for consumption in, any known or otherwise suitable oral product form consistent with the forms described herein. For example, any solid, liquid, semi-solid, semi-liquid, or powder product form, including combinations or variations thereof, are suitable for use herein, provided that such forms allow for safe and effective delivery to the individual via oral administration, of the ingredients as also defined herein.

In accordance with certain embodiments, the nutritional composition is a solid nutritional product. Non-limiting examples of solid nutritional products include snack and meal replacement products, including those formulated as bars; sticks; cookies, breads, cakes, or other baked goods; frozen liquids; candy; breakfast cereals; powders, granulated solids, or other particulates; snack chips or bites; frozen or retorted entrees; and so forth. In certain of the preceding embodiments, when the nutritional composition is a solid nutritional product, the serving is within a range of 25 grams (g) to 200 g.

In accordance with certain embodiments, the nutritional composition is a liquid nutritional composition. Non-limiting examples of liquid nutritional compositions include snack and meal replacement products; hot or cold beverages; carbonated or non-carbonated beverages; juices or other acidified beverages; milk or soy-based beverages; shakes; coffees; teas; and so forth. The liquid nutritional compositions can be formulated as suspensions or emulsions, or the liquid nutritional compositions can also be formulated in any other suitable forms such as clear liquids, solutions, liquid gels, liquid yogurts, and so forth.

When the nutritional composition is a liquid nutritional product, the serving is within a range of 30 milliliters (mL) to 500 mL (˜1 fluid ounce or fl oz to ˜17 fl oz), including from 110 mL to 500 mL (˜3.7 fl oz to ˜17 fl oz), including from 110 mL to 417 mL (˜3.7 fl oz to ˜14 fl oz), including from 120 mL to 500 mL (˜4 fl oz to ˜17 fl oz), including from 120 mL to 417 mL (˜4 fl oz to ˜14 fl oz), including from 177 mL to 417 mL (˜6 fl oz to ˜14 fl oz), including from 207 milliliters to 296 milliliters (˜7 fl oz to ˜10 fl oz), including from 230 mL to 245 mL (˜7.7 fl oz to 8.2 fl oz), including from 110 mL to 245 mL (˜3.7 fl oz to ˜8.2 fl oz), including from 120 mL to 245 mL (˜4 fl oz to ˜8.2 fl oz), including from 110 mL to 150 mL (˜3.7 fl oz to ˜5 fl oz), including from 120 mL to 150 mL (˜4 fl oz to ˜5 fl oz), including about 110 mL (˜3.7 fl oz), including about 177 mL (˜6 fl oz), including about 237 mL (˜8 fl oz), including about 296 mL (˜10 fl oz), including about 355 mL (˜12 fl oz), and including about 417 mL (˜14 fl oz).

As mentioned above, in accordance with certain embodiments, the nutritional compositions disclosed herein include at least one of a source of protein, a source of carbohydrate, a source of fat, and combinations thereof. In certain embodiments, in which the nutritional composition contains a source of protein, the source of protein is present in the nutritional composition in an amount sufficient to provide 5 g to 50 g of protein per serving, including from 6 g to 45 g and from 10 g to 30 g of protein per serving.

Any source of protein may be used so long as it is suitable for nutritional compositions and is otherwise compatible with any other selected ingredients or features in the nutritional composition. For example, the at least one source of protein may include, but is not limited to, intact, hydrolyzed, and partially hydrolyzed protein, which may be derived from any known or otherwise suitable source such as milk (e.g., casein, whey), animal (e.g., meat, fish), cereal (e.g., rice, corn), vegetable (e.g., soy, potato, pea), and combinations thereof. The at least one source of protein may also include a mixture amino acids (often described as free amino acids) known for use in nutritional products or a combination of such amino acids with the intact, hydrolyzed, and partially hydrolyzed proteins described herein. The amino acids may be naturally occurring or synthetic amino acids.

Examples of suitable sources of protein for use in the nutritional compositions disclosed herein include, but are not limited to, whey protein concentrates, whey protein isolates, whey protein hydrolysates, acid caseins, sodium caseinates, calcium caseinates, potassium caseinates, casein hydrolysates, milk protein concentrates, milk protein isolates, milk protein hydrolysates, nonfat dry milk, condensed skim milk, soy protein concentrates, soy protein isolates, soy protein hydrolysates, pea protein concentrates, pea protein isolates, pea protein hydrolysates, collagen proteins, potato proteins, rice proteins, insect proteins, earthworm proteins, fungal (e.g., mushroom) proteins, proteins expressed by microorganisms (e.g., bacteria and algae), and the like, as well as combinations thereof. The nutritional compositions can include any individual source of protein or a combination of two or more the various sources of protein listed above or otherwise encompassed by the general inventive concepts.

In certain embodiments, when the nutritional composition is a liquid and has a pH ranging from 2 to 5 such as typically found in clear nutritional liquids, the protein is limited to proteins that are soluble in an aqueous composition at this pH level. Examples of such proteins soluble in an aqueous composition at a pH of 2 to 5, include, but are not limited to, sources of whey-based proteins such as whey protein concentrates, whey protein isolates including either acidified or non-acidified whey protein isolates, whey protein hydrolysates; certain soy-based proteins such as acidified soy protein isolates and soy protein hydrolysates; certain casein-based proteins such as casein hydrolysates; certain pea-based proteins such as pea hydrolysates; the like; and combinations thereof.

In accordance with certain embodiments in which the composition is a nutritional composition and contains a source of carbohydrate, the source of carbohydrate is present in an amount sufficient to provide the nutritional composition 15 g to 110 g of carbohydrate per serving, including from 25 g to 90 g and from 40 g to 65 g of carbohydrate per serving.

Carbohydrates suitable for use in the nutritional compositions disclosed herein may be simple, complex, variations, or combinations thereof. Any source of carbohydrate may be used so long as it is suitable for use in nutritional compositions and is otherwise compatible with any other selected ingredients or features present in the nutritional composition. Non-limiting examples of a source of carbohydrate suitable for use in the nutritional compositions disclosed herein include maltodextrin; hydrolyzed or modified starch or cornstarch; glucose polymers; corn syrup; corn syrup solids; rice-derived carbohydrates; high fructose corn syrup; honey; sugar alcohols, such as maltitol, erythritol, sorbitol, glycerine, and the like; sucrose; glucose; fructose; lactose; isomaltulose, sucromalt, pullulan, potato starch, and other slowly-digested carbohydrates; oligosaccharides such as fructo-oligosaccharides; dietary fibers including, but not limited to, oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low and high methoxy pectin, cereal beta-glucans (e.g., oat beta-glucan, barley beta-glucan), carrageenan and psyllium, soluble dietary fibers such as digestion resistant maltodextrins (e.g., Fibersol™), resistant modified food starches (e.g., Actistar™), and other resistant starches; soluble and insoluble fibers derived from fruits or vegetables; and combinations thereof. The nutritional compositions can include any individual source of carbohydrate or a combination of two or more of the various sources of carbohydrate listed above or otherwise encompassed by the general inventive concepts.

In accordance with certain embodiments, the nutritional compositions may include carbohydrates that provide the composition with beneficial glycemic control and glycemic response, as well as glucose modulation. The subject's risk of developing type 2 diabetes may be reduced by consumption of such nutritional compositions. In accordance with certain embodiments, the nutritional composition has a glycemic index (GI) from 10 to 69, including from 10 to 50, including less than or equal to 50. One skilled in the art would be able to select the appropriate carbohydrates to include in the nutritional compositions disclosed herein or otherwise contemplated so as to obtain the aforementioned glycemic index.

In accordance with certain embodiments in which the composition is a nutritional composition and contains a source of fat, the source of fat is present in an amount sufficient to provide the nutritional composition 2 g to 45 g of at least one source of fat per serving, including form 5 g to 35 g and from 10 g to 30 g of fat per serving. In certain embodiments of the nutritional composition containing at least one source of fat, the nutritional composition is in the form of a liquid emulsion.

Any source of fat may be used so long as it is suitable for use in nutritional compositions and is otherwise compatible with any other selected ingredients or features present in the nutritional composition. Typically, the nutritional compositions disclosed herein that contain fat are liquid emulsions, particularly aqueous emulsions, having a pH ranging from 5 to 8, including a pH of 6 to 7, and including a pH of 6.6 to 7. The source of fat may be derived from plants, animals, and combinations thereof. Non-limiting examples of suitable sources of fat for use in the nutritional compositions described herein include coconut oil, fractionated coconut oil, soy oil, corn oil, olive oil, safflower oil, high oleic safflower oil, medium chain triglyceride (MCT) oil, high gamma linolenic (GLA) safflower oil, sunflower oil, high oleic sunflower oil, palm oil, palm kernel oil, palm olein, canola oil, marine oils, fish oils such as those containing from 40% to 70% by weight of a combination of eicosapentaenoic acid and docosahexaenoic acid, algal oils, transgenic oils, cottonseed oils, interesterified oils, transesterified oils, eicosapentaenoic acid, docosahexaenoic acid, and the like, as well as combinations thereof. The nutritional compositions can include any individual source of fat or a combination of two or more of the various sources of fat listed above or otherwise encompassed by the general inventive concepts.

In certain embodiments of the methods disclosed herein in which the composition is a nutritional composition, the nutritional composition may contain a limited amount of fat. The limited amount of fat may be due at least in part to the desired clarity, desired pH, or both desired clarity and desired pH of the liquid nutritional composition. Typically, the liquid nutritional compositions desired to be clear, or at least substantially translucent, are substantially free of fat. As used herein “substantially free of fat” refers to nutritional compositions containing less than 0.5%, and including less than 0.1% by weight solids of fat based on the total weight of the composition. “Substantially free of fat” also may refer to nutritional compositions disclosed herein that contain no fat, i.e., zero fat. Furthermore, those liquid nutritional compositions that have a desired acidic pH in the range of 2 to 5, e.g., juices, fruit juices, fruit-flavored beverages, etc., typically are substantially free of fat. Liquid nutritional compositions that are both clear and have a pH ranging from 2 to 5 are also typically substantially free of fat. In certain of the preceding embodiments, the nutritional composition is a clear liquid nutritional product having a pH of 2 to 5 and having no more than 0.5 weight % fat based on the total weight of the nutritional composition. In accordance with certain embodiments, the pH of the nutritional composition may be from 2.5 to 4.6, including a pH of 3 to 3.5. In those embodiments of the nutritional compositions that are substantially free of fat but have some amount of fat present, the fat may be present as a result of being inherently present in another ingredient (e.g., a source of protein), may be present as a result of being added as one of more separate sources of fat, or a combination thereof.

The amount or concentration of the at least one of a source of protein, source of carbohydrate, and source of fat present in the nutritional compositions may vary widely depending on the product formulation of the nutritional composition (e.g., clear liquid, fat-based emulsion). In addition, the amount or concentration of the at least one of a source of protein, source of carbohydrate, and source of fat may be characterized based upon a percentage of the total calories per serving in the nutritional composition. For example, in certain embodiments, the amount or concentration of the at least one of a source of protein, source of carbohydrate, and source of fat present in the nutritional composition can be within the ranges described in Samples A-E, as shown in the Table 1 below.

TABLE 1

| Nutrient (% total | Sample | Sample | Sample | Sample | Sample |
|---|---|---|---|---|---|
| calories) | A | B | C | D | E |
| Carbohydrate | 0-94 | 20-85 | 35-55 | 45-65 | 75-85 |
| Fat | 0-94 | 5-50 | 15-35 | 25-35 | 0-5 |
| Protein | 6-100 | 10-50 | 20-50 | 10-30 | 15-25 |

In certain embodiments, the nutritional compositions may include other compounds or sources of such compounds that are anabolic for muscle, stimulate muscle protein synthesis, decrease muscle protein degradation, or combinations thereof. Examples of such compounds include, but are not limited to, leucine, isoleucine, valine, glycine, histidine, lysine, methionine, phenylalanine, threonine, tryptophan, carnitine, carnosine, creatine, taurine, arginine, anserine, mushroom extract, cordycepic acid, spinach extract, arugula extract, broccoli extract, eggplant skin extract, plum extract, apple extract, ursolic acid, grape extract, resveratrol, bioidentical stilbenes such as pTeroPure™, olive extract, alpha-ketoisocaproic acid, alpha-hydroxyisocaproic acid, and metabolites of any of the foregoing. In accordance with one or more embodiments, the nutritional compositions include at least one source of a compound selected from the group consisting of leucine, isoleucine, valine, glycine, histidine, lysine, methionine, phenylalanine, threonine, tryptophan, carnitine, carnosine, creatine, and metabolites of any of the foregoing, as well as combinations thereof.

A non-limiting example of a suitable metabolite of leucine is beta-hydroxy-beta-methylbutyrate (HMB). Suitable sources of HMB include HMB as the free acid, a salt, an anhydrous salt, an ester, a lactone, or other product forms that otherwise provide a bioavailable form of HMB in the nutritional product. Non-limiting examples of suitable salts of HMB for use herein include HMB salts, hydrated or anhydrous, of sodium, potassium, magnesium, chromium, calcium, or other non-toxic salt form. In certain of the embodiments containing HMB, the nutritional composition may comprise 0.5 g to 3.5 g of HMB per serving, including 0.5 g to 3 g of HMB per serving, and including 0.5 g to 1.5 g of HMB per serving.

In certain embodiments, the nutritional compositions may also include at least one food-grade acid. As discussed above, certain of the nutritional compositions disclosed herein have a pH of 2 to 5, and in certain of the preceding embodiments, a pH of 2.5 to 4.6 or a pH of 3 to 3.5. The food-grade acid may be added to the nutritional composition to adjust the pH of the overall nutritional composition to obtain a pH within a desired range, such as a pH from 2 to 5, a pH from 2.5 to 4.6, or a pH from 3 to 3.5. Any suitable food-grade acid that is capable of adjusting the pH of the nutritional composition to a pH ranging from 2.5 to 4.6, or a pH ranging from 3 to 3.5 may be used. Non-limiting examples of such suitable food-grade acids include citric acid, acetic acid, lactic acid, maleic acid, ascorbic acid, phosphoric acid, hydrochloric acid, and the like.

The amount or concentration of the food-grade acid required to obtain the intended pH depends on various factors, such as the initial pH of the finalized formulation, the relative strength or weakness of the selected food-grade acid, the concentration of the selected food-grade acid, the quantity of the nutritional composition, etc. The type of acid selected may also be based on the type of flavor desired in the nutritional composition, e.g., for a lemon flavored product, citric acid is more suitable, while for the apple flavored product, maleic acid is more suitable. In addition, the pH can also be adjusted by addition of clear juices, e.g., cranberry, lemon juice, lime juice, pineapple juice, and the like, including mixtures and combinations thereof, which can be added to adjust the pH to desired levels. Furthermore, in the case of the over-addition of acid, a suitable food grade base, e.g., sodium hydroxide, calcium hydroxide, potassium hydroxide and the like, can be used to bring the pH of the nutritional composition to the desired level.

In certain embodiments, the nutritional compositions may include a high intensity sweetener to counter, mask, or otherwise obscure the potent taste of the green tea extract, particularly the EGCg present in the green tea extract, which may be described as sour, astringent, and bitter, as well as to counter, mask, or otherwise obscure the taste of any of the other polyphenols in the green tea extract that may be present in the composition. Examples of suitable high intensity sweeteners include, but are not limited to, sucralose, acesulfame potassium (also known as “acesulfame K” or “ace K”), aspartame, stevia, neotame, neohesperidine DC, alitame, monellin, thaumatin, mogrosides, monk fruit, and the like. Combinations of the high intensity sweeteners listed above may be used. The amount of the high intensity sweetener in the nutritional composition may vary depending upon the particular high intensity sweetener selected, other ingredients in the formulation, and other formulation or product target variables. Different high intensity sweeteners themselves have different sweetness intensities (e.g., acesulfame K is approximately 200 times sweeter than sucrose as compared to sucralose which is approximately 600 times sweeter than sucrose), and therefore may require more or less sweetener relative to other sweeteners. Furthermore, certain carbohydrates, which may already be present in the nutritional compositions disclosed herein, are sweeteners that may at least partially counter or at least partially mask the taste of the green tea extract in such nutritional compositions.

In certain embodiments, the nutritional compositions may comprise a viscosity agent, e.g., thickening agent. Typically, the viscosity agent is used in the thicker types of liquid nutritional compositions, e.g., the fat-based emulsions, shakes, etc. Any viscosity agent that is known or otherwise suitable for use in a nutritional composition is also suitable for use herein, some non-limiting examples of which include starches, such as modified corn starch, wheat starch (including pregelatinized wheat starch), potato starch, rice starch, tapioca starch, and the like; blends of cellulose gel and cellulose gum; blends of microcrystalline cellulose and sodium carboxymethyl cellulose; pectin; carrageenan; agar; gellan gum; alginates; gum acacia; gelatin; methyl cellulose; hydroxypropylcellulose; and combinations thereof. In certain embodiments, the viscosity agent is present in an amount of 0 to about 5.0%, including from about 0.1% to about 3%, including from about 0.5% to about 1.5%, by weight solids based on the total weight of the nutritional composition.

Furthermore, the nutritional compositions disclosed herein or otherwise encompassed by the general inventive concepts may also contain other ingredients, non-limiting examples of which include, preservatives, antioxidants in addition to those found in the green tea extract, buffers, pharmaceutical actives, additional nutrients, colorants, flavors, emulsifiers, anti-foam agents, and the like.

In certain embodiments, the nutritional compositions may also contain vitamins or related nutrients including, but not limited to, curcumin, lutein, fish oil, vitamin A, vitamin D (cholecalciferol, 25-hydroxycholecalciferol, 1,25-dihydroxycholecalciferol, 24,25-dihydroxycholecalciferol, ergocalciferol), vitamin E, vitamin K1, vitamin K2, thiamine, riboflavin, pyridoxine, vitamin B12, carotenoids, niacin, folic acid, pantothenic acid, biotin, vitamin C, choline, inositol, salts, derivatives thereof, and combinations thereof.

Additionally, in accordance with certain embodiments, the nutritional compositions disclosed herein may also contain minerals including, but not limited to, phosphorus, magnesium, iron, manganese, copper, sodium, potassium, molybdenum, chromium, selenium, chloride, zinc, and combinations thereof.

The various embodiments of the nutritional compositions disclosed herein or otherwise encompassed by the general inventive concepts may also be substantially free of any optional ingredient or feature described herein, provided that the remaining nutritional composition still contains all of the required ingredients or features as described herein. In this context, and unless otherwise specified, the term “substantially free” means that the selected nutritional compositions contain less than a functional amount of the optional ingredient, typically less than 0.5% by weight solids of such optional ingredient based on the total weight of the nutritional composition, including less than 0.1% and also including zero, by weight solids of such optional ingredient based on the total weight of the nutritional composition.

Exemplary formulations of liquid nutritional compositions in accordance with various exemplary embodiments disclosed herein are provided below in Tables 2 and 3. All ingredient amounts listed in Tables 2 and 3 are listed as kilogram per 1,000 kg batch of product, unless otherwise indicated.

Table 2 shows an exemplary formulation of an emulsion-type liquid nutritional composition containing fat, protein, and carbohydrates and having a pH in the range of 6.6 to 7. Assuming a density of 1.075 g/mL and a serving size of about 237 mL (˜8 fl oz), a nutritional composition made according to the formulation shown in Table 2 has about 177 mg of EGCg per serving.

TABLE 2

| INGREDIENTS | Amount (kg/1,000 kg) |
|---|---|
| Water | Quantity Sufficient |
| Green Tea Extract¹ | 1.390 |
| Sucrose | 89.1 |
| Maltodextrin | 69.1 |
| Milk Protein Concentrate | 38.6 |
| Soy Oil | 13.3 |
| Canola Oil | 5.3 |
| Soy Protein Concentrate | 4.7 |
| Corn Oil | 4.1 |
| Potassium Citrate | 2.7 |
| Natural and artificial Vanilla Flavor | 2.0 |
| Magnesium Phosphate Dibasic | 1.9 |
| Sodium Citrate | 1.6 |
| Soy Lecithin | 1.4 |
| Tricalcium Phosphate | 1.3 |
| Magnesium Chloride | 1.2 |
| Sodium Chloride | 0.718 |
| Choline Chloride | 0.480 |
| Ascorbic Acid | 0.469 |
| Carrageenan | 0.450 |
| Ultra Trace Mineral/Trace Mineral Premix | 0.364 |
| Potassium Hydroxide (Processing aid) | 0.323 |
| Potassium Chloride | 0.308 |
| Vitamin Premix² | 0.1465 |
| Potassium Iodide | 0.000207 |

¹SUNPHENON ® 90D (available from Taiyo International, Inc. of Minneapolis, Minnesota) is a green tea extract that contains approximately 50% by weight of EGCg, i.e., 1.390 kg of green tea extract contains approximately 0.695 kg EGCg.
²Vitamin premix includes one or more of the following: dl-Alpha-Tocopheryl Acetate, Vitamin A Palmitate, Phylloquinone, Vitamin D3, Niacinamide, d-Calcium Pantothenate, Thiamine Chloride Hydrochloride, Pyridoxine Hydrochloride, Riboflavin, Folic Acid, Biotin, Cyanocobalamin, etc.

Table 3 shows an exemplary formulation of a clear-type liquid nutritional composition that is substantially free of fat and has a pH in the range of 3 to 3.5. Assuming a density of 1.05 g/mL and a serving size of about 296 mL (˜10 fl oz), a nutritional composition made according to the formulation shown in Table 3 has about 188 mg of EGCg per serving.

TABLE 3

| INGREDIENTS | Amount (kg/1,000 kg) |
|---|---|
| Water | Quantity Sufficient |
| Sucrose | 50.7 |
| Corn syrup solids | 61.3 |
| Acidified Whey Protein Isolate | 35.7 |
| Citric Acid | 2.00 |
| Flavoring | 2.00 |
| Green Tea Extract¹ | 1.212 |
| Ascorbic Acid | 0.535 |
| Liquid Sucralose (25%) | 0.275 |
| Ultra Trace Mineral/Trace Mineral Premix | 0.230 |
| Vitamin Premix² | 0.219 |
| Acesulfame Potassium | 0.110 |
| Antifoam processing aid (non-silicone) | 0.060 |
| Coloring | 0.0589 |
| Natural and Artificial Peach Flavor | 2.0 |
| Folic Acid | 0.0013 |
| Potassium Iodide | 0.000204 |

¹SUNPHENON ® 90D, which is a green tea extract that contains approximately 50% by weight of EGCg, i.e., 1.212 kg of green tea extract contains approximately 0.606 kg EGCg.
²Vitamin premix includes one or more of the following: dl-Alpha-Tocopheryl Acetate, Vitamin A Palmitate, Phylloquinone, Vitamin D3, Niacinamide, d-Calcium Pantothenate, Thiamine Chloride Hydrochloride, Pyridoxine Hydrochloride, Riboflavin, Folic Acid, Biotin, Cyanocobalamin, etc.

The various embodiments of exemplary nutritional compositions disclosed herein may be prepared by any process or suitable method (now known or known in the future) for making the selected product form, such as a liquid or semi-liquid nutritional composition.

In one suitable manufacturing process for preparing emulsion-type liquid nutritional compositions (e.g., the composition listed in Table 2 above), at least three separate slurries are prepared, including a protein-in-fat (PIF) slurry, a carbohydrate-mineral (CHO-MIN) slurry, and a protein-in-water (PIW) slurry. The PIF slurry is formed by heating and mixing an oil (e.g., soy oil, canola oil, corn oil) and then adding an emulsifier (e.g., lecithin), fat soluble vitamins, and a portion of the total protein (e.g., milk protein concentrate) with continued heat and agitation. The CHO-MIN slurry is formed by adding with heated agitation to water: minerals (e.g., potassium citrate, dipotassium phosphate, sodium citrate), including trace and ultra trace minerals (Ultra Trace Mineral/Trace Mineral Premix), and thickening or viscosity agents (e.g., cellulose gel, gellan, carrageenan). The resulting CHO-MIN slurry is held for 10 minutes with continued heat and agitation before adding additional minerals (e.g., potassium chloride, magnesium carbonate, potassium iodide) and the carbohydrates (e.g., sucrose, corn syrup). The PIW slurry is then formed by mixing with heat and agitation the remaining protein.

In accordance with this exemplary process, the three slurries are blended together with heat and agitation and the pH is adjusted to the desired range, e.g., from 6.6 to 7, after which the composition is subjected to high-temperature short-time (“HTST”) processing. The composition is heat treated, emulsified, homogenized, and cooled during HTST. Water soluble vitamins and ascorbic acid are added (if applicable), the pH is again adjusted (if necessary), flavors are added and any additional water can be added to adjust the solids content to the desired range. The green tea extract is prepared as a solution (e.g., 1% w/w) by adding to water and agitating for 0-24 hours. The solution of green tea extract is added to the composition containing the other ingredients and is agitated for a period of time, e.g., 5 to 60 minutes, to ensure homogeneous distribution of the green tea extract in the composition. The agitation associated with the preparation of the solution containing the green tea extract, as well as the agitation associated with the addition of such green tea extract solution to the other ingredients, may take place at 4° C. to 50° C. At this point, the liquid nutritional composition may optionally be packaged and sterilized according to any suitable sterilization technique (e.g., aseptic, retort, hot-fill, chemical, radiation, and filtering sterilization techniques).

In another exemplary manufacturing process which is suitable for preparing clear-type or more acidic-type liquid nutritional compositions (e.g., the composition listed in Table 3 above), protein is added to room temperature water in a kettle. The protein and water mixture is agitated until all of the protein dissolves into solution and before adding any other ingredients. All the other ingredients, except the green tea extract and food-grade acids are then added in the kettle. Before the green tea extract is added, the pH of the blend is adjusted to the target pH, e.g., a pH of 3 to 3.5, with the food-grade acids. The solution of the green tea extract is prepared in a separate kettle in the same manner as described above. The green tea extract solution is then added to the kettle containing the other ingredients and is agitated for a period of time, e.g., 5 to 60 minutes, to ensure homogeneous distribution of the green tea extract in the composition. The agitation associated with the preparation of the solution containing the green tea extract, as well as the agitation associated with the addition of such green tea extract solution to the other ingredients, may take place at 4° C. to 50° C. At this point, the liquid nutritional composition may optionally be packaged and sterilized according to any suitable sterilization technique (e.g., aseptic, retort, hot-fill, chemical, radiation, and filtering sterilization techniques).

In accordance with certain embodiments, the methods disclosed herein increase skeletal muscle protein synthesis in a subject without a concomitant increase in food intake by the subject. In other words, the subject increases skeletal muscle protein synthesis without simultaneously increasing his or her food intake on top of, or in addition to, the administration of the green tea extract according to the methods disclosed herein. Unless otherwise indicated herein, the term “food intake” refers to the weight (mass) of food consumed by the subject in addition to the composition containing the green tea extract. Thus, the subject can increase skeletal muscle protein synthesis without adjusting the total amount of food (by weight) consumed in addition to the administration of the green tea extract as disclosed herein. Without intending to be limited by any theory, it is believed that this increase in skeletal muscle protein synthesis results from an improved energy utilization and compartmentalization at a cellular level, and alternatively or in addition, increases the percentage of nutrients utilized by the musculature, resulting from the administration of the green tea extract.

### EXAMPLES

The following examples illustrate specific and exemplary embodiments and/or features of the methods disclosed herein. The examples are provided solely for the purposes of illustration and should not be construed as limitations of the present disclosure. Numerous variations over these specific examples are possible without departing from the spirit and scope of the presently disclosed methods.

### Example 1

The role of EGCg, in the form of TEAVIGO® brand green tea extract that contains about 95% EGCg (available from DSM of the Netherlands), in stimulating muscle protein synthesis was evaluated in an in vitro cell-based assay using mouse C₂C₁₂ muscle myotubes. This assay measured the incorporation of radiolabeled phenylalanine into myotubes in response to various stimuli.

C₂C₁₂ myoblasts passaged in FCS DMEM (Fetal Calf Serum Dulbecco's Modified Eagle's Medium) were differentiated to myotubes in HS DMEM (Horse Serum Dulbecco's Modified Eagle's Medium). The myotubes were labeled for 24 hours with L-[2,6-3H] phenylalanine prior to compound experimentation. They were then incubated with TNF-α (50 ng/ml) for 2 hours in the absence, or presence of 10 μM or 50 μM of EGCg, and protein synthesis was measured over the following 4 hours.

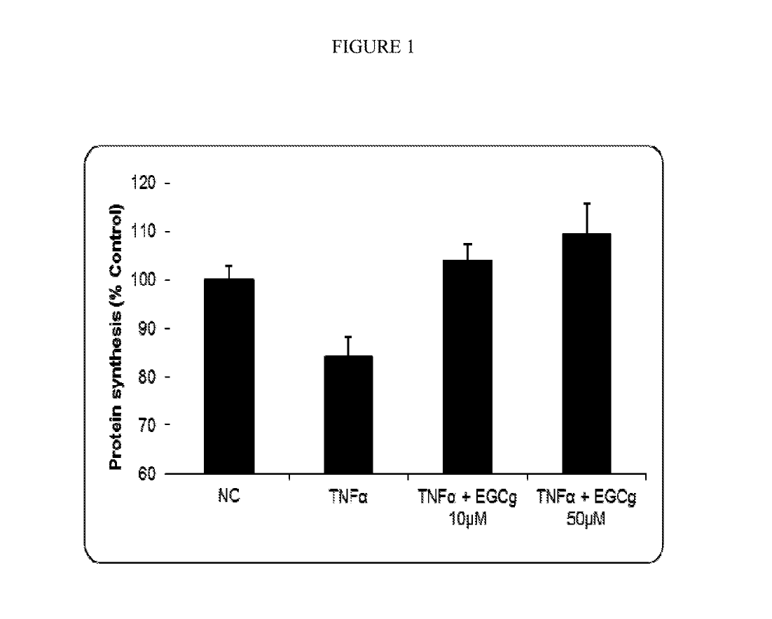

As shown in FIG. 1, TNFα alone presented a significant stressor, and was associated with a significant 16% drop in protein synthesis (p<0.05) as compared to the control, i.e., NC (Normal Control) in FIG. 1, which was not subjected to the TNFα. The presence of 10 μM and 50 μM of EGCg in addition to the TNFα stressor resulted in protein synthesis at 104% (p<0.05) and 109% (p<0.01), respectively, as compared to the control, thereby demonstrating a significant recovery from the effects of TNFα.

Accordingly, based on this assay, EGCg was shown to recover muscle protein synthesis in a dose dependent manner in myotubes exposed to TNFα. While exposure to TNFα resulted in a 16% reduction in protein synthesis versus the control, the additional exposure to 10 μM or 50 μM EGCg was protective against this effect, with protein synthesis rates, as mentioned above, at 104% and 109%, respectively, of the control.

### Example 2

Each of 12 aged Sprague Dawley rats (SD) rats (20 months) were chronically fed the following diets ad libitum for 8 weeks to evaluate the effects on the progression of sarcopenia in the rats:

Control diet: AIN-93M (AIN-93M diets are well known balanced diets for rodents); and

Test diet: AIN-93M+200 mg/kg body weight of EGCg.

The AIN-93M diet was supplemented with EGCG (in the form of TEAVIGO® brand green tea extract) at a concentration of 5 gm/kg diet to deliver a dose of 200 mg/kg body weight of EGCg per day to the SD rat (approximate body weight 500 gm, approximate food intake=20 gm/d) in the test diet.

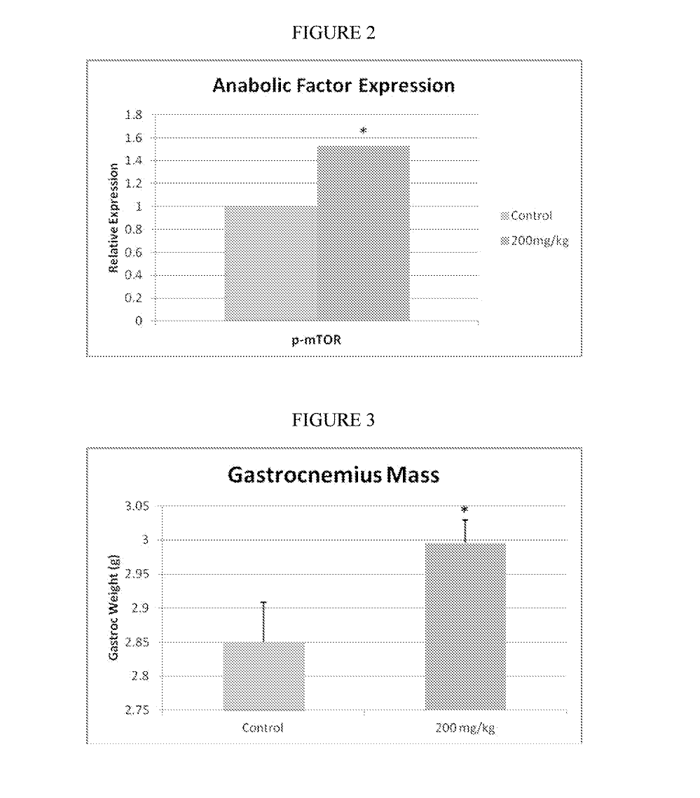

EGCg treatment significantly increased the activation of mTOR in skeletal muscle by 53%, as evidenced by a 53% increase in intramuscular phosphorylated mTOR expression in SD rats fed the test diet (EGCg treatment) as compared to the SD Rats fed the control diet. These results are shown in FIG. 2 (p<0.05).

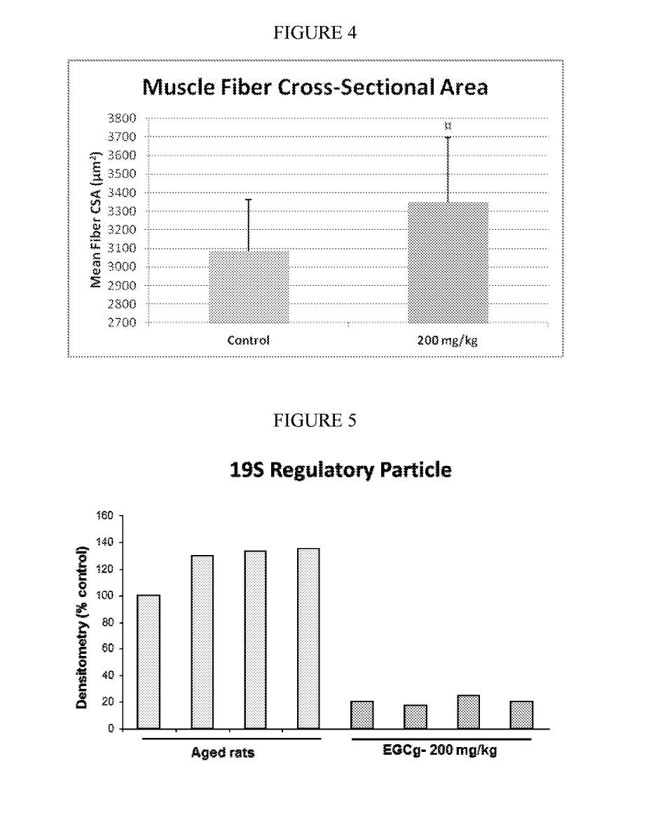

This increased activation was associated with increases in gastrocnemius muscle wet weight and muscle fiber cross sectional area, as shown respectively in FIGS. 3 and 4. Excised gastrocnemius muscles were weighed at the end of the 8 weeks. FIG. 3 shows approximately 5% greater muscle mass in excised gastrocnemius muscle from SD rats fed the test diet (EGCg treatment) as compared to excised gastrocnemius muscle from SD rats fed the control diet. With respect to the muscle fiber cross sectional area, 8-micron muscle sections from the SD rats were dissected, mounted, and stained using hematoxylin & eosin at the end of the 8 week period. Approximately 400 fibers were measured per sample. The results are shown in FIG. 4, in which the SD rats fed the test diet (EGCg treatment) have higher cross sectional areas as compared to that of the SD rats fed the control diet (p=0.06).

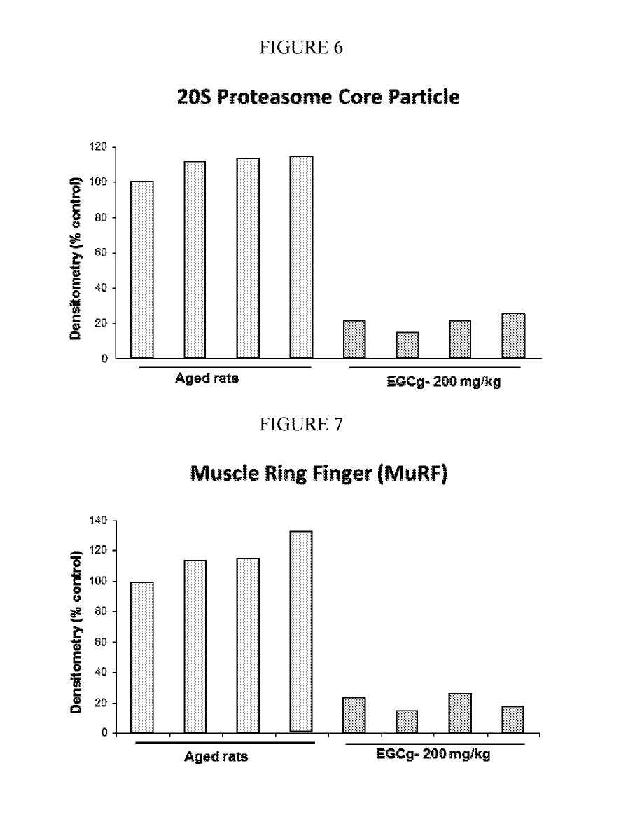

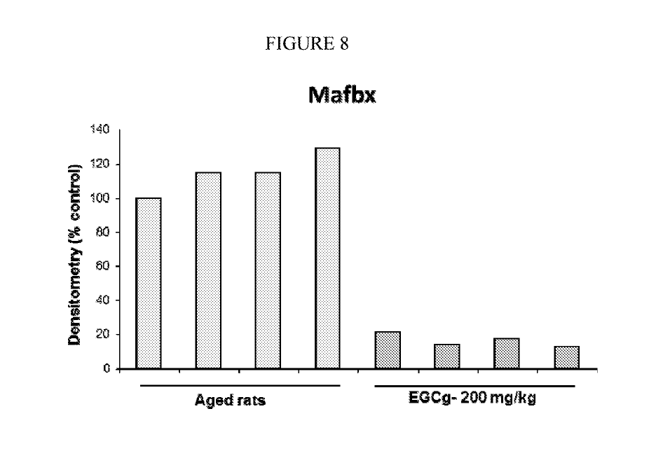

Conversely, markers of muscle atrophy such as the 19S regulatory and 20S core particle ubiquitin proteasome subunits, MuRF1, and MAFbx were lower at the end of the 8 weeks in those SD rats fed the test diet (EGCg treatment) as compared to those fed the control diet. FIGS. 5-8, which respectively show the 19S regulatory particle ubiquitin proteasome subunits, 20S core particle ubiquitin proteasome subunits, MuRF1, and MAFbx of SD rats fed either the test diet (EGCg treatment) or the control diet, show lower expression for each of these markers for the SD rats fed the test diets as compared to those fed the control. The components of the Ubiquitin Proteasome Pathway (UPP) as shown in FIGS. 5-8 were measured by western blotting in rats treated with either the test (EGCg treatment) or the control diets.

Unless otherwise indicated herein, all sub-embodiments and optional embodiments are respective sub-embodiments and optional embodiments to all other embodiments described herein. While the present application has been illustrated by the description of various exemplary embodiments of the general inventive concepts, and while the exemplary embodiments have been described in considerable detail, it is not the intention of the applicants to restrict or in any way limit the scope of the appended claims or general inventive concepts to such detail. Additional advantages and modifications will readily appear to those skilled in the art. Therefore, the application, in its broader aspects, is not limited to the specific details, the representative apparatus, and illustrative examples shown and described. Accordingly, departures may be made from such details without departing from the spirit or scope of the general inventive concepts as taught and suggested by the disclosure herein.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Also, to the extent that the terms “in” or “into” are used in the specification or the claims, it is intended to additionally mean “on” or “onto.”

As used in the description of the invention and the appended claims, the singular forms “a,” “an,” and “the” are intended to include the plural forms as well, unless the context clearly indicates otherwise.

1. A composition for use in increasing skeletal muscle protein synthesis in a subject, wherein the composition comprises 20 mg to 2,000 mg of a green tea extract per serving and wherein the dosage is at least one serving per day.

2. The composition according to claim 1, wherein skeletal muscle protein synthesis increases under stress caused by at least one condition or agent that inhibits skeletal muscle protein synthesis.

3. The composition according to claim 1, wherein the green tea extract contains 30% to 100% by weight solids of epigallocatechin gallate (EGCg).

4. (canceled)

5. The composition according to claim 1, wherein the composition further comprises at least one of a catechin (C), a gallocatechin (GC), an epicatechin (EC), an epicatechin gallate (ECg), an epigallocatechin (EGC), and combinations thereof.

6. The composition according to claim 1, wherein the composition is a nutritional composition.

7. The composition according to claim 1, wherein the composition further comprises 0.5 g to 3 g per serving of beta-hydroxy-beta-methyl butyrate (HMB).

8. (canceled)

9. The composition according to claim 1, wherein the composition further comprises a source of protein comprising at least one of a whey protein concentrate, a whey protein isolate, a whey protein hydrolysate, an acid casein, a sodium caseinate, a calcium caseinate, a potassium caseinate, a casein hydrolysate, a milk protein concentrate, a milk protein isolate, a milk protein hydrolysate, nonfat dry milk, condensed skim milk, a soy protein concentrate, a soy protein isolate, a soy protein hydrolysate, a pea protein concentrate, a pea protein isolate, a pea protein hydrolysate, a collagen protein, potato protein, rice protein, and combinations thereof.

10. The composition according to claim 1, wherein the composition further comprises a source of carbohydrate comprising at least one of maltodextrin, hydrolyzed or modified starch or cornstarch, glucose polymer, corn syrup, corn syrup solid, rice-derived carbohydrate, sucrose, glucose, fructose, lactose, high fructose corn syrup, honey, sugar alcohol, and combinations thereof.

11. The composition according to claim 1, wherein the source of fat comprises at least one of coconut oil, fractionated coconut oil, soy oil, corn oil, olive oil, safflower oil, high oleic safflower oil, medium chain triglyceride oil, high gamma linolenic safflower oil, sunflower oil, high oleic sunflower oil, palm oil, palm kernel oil, palm olein, canola oil, marine oils, fish oils, algal oils, transgenic oils, cottonseed oils, interesterified oils, transesterified oils, eicosapentaenoic acid, docosahexaenoic acid, and combinations thereof.

12. The composition according to claim 6, wherein the nutritional composition is a clear liquid nutritional product having a pH of 2 to 5, wherein the nutritional composition has at least one of a source of protein and a source of carbohydrate, and wherein the nutritional composition has no more than 0.5% by weight solids of fat based on the total weight of the nutritional composition.

13. The composition according to claim 6, wherein a glycemic index of the nutritional composition is less than or equal to 50.

14.-16. (canceled)

17. The composition according to claim 1, wherein the subject is susceptible to skeletal muscle loss attributable to age, malnourishment, disease, injury, infection, hospitalization, lack of appetite, lack of mobility, medication, and combinations thereof.

18. The composition according to claim 1, wherein the subject experiences skeletal muscle loss attributable to age, malnourishment, disease, injury, infection, hospitalization, lack of appetite, lack of mobility, medication, and combinations thereof.

19. The composition according to claim 1, wherein the skeletal muscle loss is attributable to at least one of sarcopenia, surgery, post-hospitalization rehabilitation, appetite loss, inflammation, dysphagia, cognitive impairment, impaired nutrient absorption, taste aversion, frailty syndrome, cancer cachexia, chronic obstructive pulmonary disease (COPD), end stage renal disease (ESRD), congestive heart failure (CHF), acquired immunodeficiency syndrome (AIDS), chemotherapy, acute illness, lack of use of extremities, disability, muscular dystrophy, joint disease, and combinations thereof.

20. (canceled)

21. (canceled)

22. The composition according to claim 1, wherein the subject is in need of increasing or maintaining mammalian target of rapamycin (mTOR) activation.

23. A composition for use in increasing or maintaining mammalian target of rapamycin (mTOR) activation in a subject in need thereof, wherein the composition comprises 20 mg to 2,000 mg of a green tea extract per serving, and wherein the dosage is at least one serving per day.

24. The composition according to claim 23, wherein skeletal muscle protein synthesis increases under stress caused by at least one condition or agent that inhibits skeletal muscle protein synthesis.

25. The composition according to claim 23, wherein the green tea extract contains 30% to 100% by weight solids of epigallocatechin gallate (EGCg).

26. (canceled)

27. The composition according to claim 23, wherein the composition further comprises at least one of a catechin (C), a gallocatechin (GC), an epicatechin (EC), an epicatechin gallate (ECg), an epigallocatechin (EGC), and beta-hydroxy-beta-methyl butyrate (HMB).

28. (canceled)

29.-35. (canceled)

36. A method for increasing skeletal muscle protein synthesis in a subject, the method comprising administering at least one serving per day of a composition including from 20 mg to 2,000 mg of a green tea extract per serving to the subject.

37. The method of claim 36 further comprising characterizing the subject, prior to the step of administering the at least one serving, as having age-related skeletal muscle loss due to at least one of sarcopenia, hospitalization, surgery, post-hospitalization rehabilitation, appetite loss, inflammation, dysphagia, cognitive impairment, impaired nutrient absorption, taste aversion, and frailty syndrome.

38. The method of claim 36 wherein the step of administering the at least one serving comprises enteral administration.
